(19) United States
(12) Patent Application Publication
(10) Pub. No.: US 2011/0070329 A1
(43) Pub. Date: Mar. 24, 2011

(54) 1-tert-Butylcyclohexanecarboxamide and uses thereof as cooling compounds

(76) Inventors: Arkadiusz Kazimierski, Old Bridge, NJ (US); Kenneth J. Kraut, Union Beach, NJ (US)

(21) Appl. No.: 12/562,510

(22) Filed: Sep. 18, 2009

(51) Int. Cl. A23G 4/06; A23L 1/00; A23L 2/00; A23K 1/00; A23G 3/00; C07D 211/70; C07C 233/57

(52) U.S. Cl. 426/3; 426/538; 426/535; 426/537; 546/337; 564/123

(57) ABSTRACT

Described is a new cooling agent represented by Formula I and compositions with known coolers having cooling properties and the application of Formula I in foodstuffs and chewing gum:

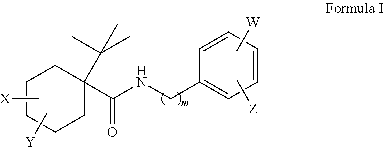

wherein X, Y is independently selected from a C1-C4 straight or branched alkyl or H atom; and m is 0, 1 or 2, Z when present is nitrogen, W and W′ are selected independently from the group consisting of H, OH, C1-C4 straight or branched alkyl, and a C1-C4 straight or branched alkoxy or W is group (CH₂)n-R, where n is 0 or 1 and R is a group with non-bonding electrons. Useful groups with non-bonding electrons are halogens, OH, OMe, OEt, NO₂, CN, Ac, SO₂NH₂, CHO, CO₂H, CONH₂, CONHR², where R² is selected from C1-C4, and C1-C4 alkyl carboxylates such as CO₂Me or CO₂Et.

### FIELD OF INVENTION

The following invention relates to edible compositions having a unique, long lasting, cooling perception which provides the user with an enhanced perception of breath-freshening without bitterness. The following invention is useful in chewing gum compositions and confectionary compositions which provide a long-lasting, breath freshening perception without bitterness.

### BACKGROUND OF THE INVENTION

Cooling agents are describes as chemical compounds providing cold or cool sensations when contacted with the human body, especially with the mucus membranes of the mouth, nose and throat. Cooling compounds are widely used in edible products, beverages, dentifrices, tobacco products, mouthwashes and toiletries.

One class of cooling compounds which are very effective compounds containing an N-substituted p-menthane carboxamide moiety. Examples of these compounds are described in, for example, British Patents GB 1,351,761-2 and U.S. Pat. No. 4,150,052.

Properties of those compounds are based on l-menthol, which is one of the most well-known physiological coolants, and which has been widely used in several applications. L-Menthol has an excellent cooling strength and is relatively inexpensive.

It is well recognized that one of the purpose of chewing gum and confectionery products is to enhance ones breath and provide a clean, cool, fresh feeling in the mouth. Unfortunately, most products are not able to maintain such perception for long periods of time, which is a time up to about thirty minutes.

The compound of the present invention may be used alone or in combination with additional compounds or composition which increase cooling and flavor impact and extend these taste effects over a prolonged period of time.

### SUMMARY OF THE INVENTION

One embodiment of the invention is directed to cooling compounds represented by Formula I

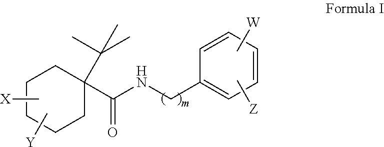

wherein X, Y is independently selected from a C1-C4 straight or branched alkyl or H atom; and m is 0, 1 or 2, Z when present is nitrogen, W and W′ are selected independently from the group consisting of H, OH, C1-C4 straight or branched alkyl, and a C1-C4 straight or branched alkoxy or W is group (CH₂)n-R, where n is 0 or 1 and R is a group with non-bonding electrons. Useful groups with non-bonding electrons are halogens, OH, OMe, OEt, NO₂, CN, acetyl group, herein referred to as Ac, SO₂NH₂, CHO, CO₂H, CONH₂, CONHR2, where R2 is selected from C1-C4, and C1-C4 alkyl carboxylates such as CO₂Me or CO₂Et.

In another embodiment of the invention a composition is provided containing the combination of Formula I with other compounds and compositions having cooling properties.

In another embodiment of the invention an ingestible product for humans or animals which comprises a product base selected from the group consisting of baked goods, dairy products, fruit ices, confectionery products, sugarless candies, jams, jellies, gelatins, puddings, animal feeds, pressed confectionery tablets, hard-boiled candies, pectin-based candies, chewy candies, creme-centered candies, fondants, sugarless hard-boiled candies, sugarless pectin-based candies, sugarless chewy candies, sugarless creme-centered candies, toothpastes, mouthwashes, breath fresheners, carbonated beverages, mineral waters, powdered beverage mixes, other non-alcoholic beverages, cough drops, lozenges, cough mixtures, decongestants, anti-irritants, antacids, anti-indigestion preparations and oral analgesics, and an effective amount of Formula I.

In accordance with yet another embodiment of the present invention, a chewing gum composition which is capable of providing long-lasting, breath freshening perception without bitterness comprises a gum base, a sweetener and a cooling composition comprising Formula I.

Another embodiment of the invention is directed to a process for augmenting, enhancing or imparting a taste or cooling or refreshing effect in or to a cosmetic, skin care product, lip gloss, hair care product, cologne, shaving balm, after-shave lotion, dairy product, fruit ice preparation, confectionery, mouth and throat lozenges, cough mixtures, decongestants, antacids, oral analgesics or other medicinal products, chewing gum, candy (including hard candy and soft candy), fondants, toothpaste, mouthwashes, mineral water, alcoholic beverage, non-alcoholic beverage, powdered beverage, or other foodstuff comprising the step of adding to a cosmetic, skin care product, lip gloss, hair care product, cologne, shaving balm, after-shave lotion, dairy product, fruit ice preparation, confectionery, lozenges, cough mixtures, decongestants, antacids, oral analgesics or other medicinal product, chewing gum, candy, fondants, toothpaste, mouthwashes, mineral water, alcoholic beverage, non-alcoholic beverage, powdered beverage, or other foodstuff, a taste or cooling or refreshing effect augmenting, enhancing or imparting quantity and concentration of Formula I.

### DETAILED DESCRIPTION OF THE INVENTION

Applicants have unexpectedly found that the cooling compounds represented by Formula I, showing strong cooling activity when used by itself or in combination with another coolers.

In specific embodiments of the invention, the following cooling compounds are disclosed:

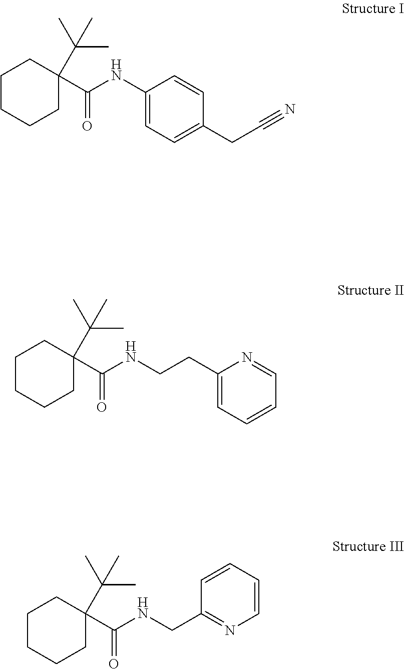

In accordance with a another embodiment of the present invention, a chewing gum composition which is capable of providing long-lasting, breath freshening perception without bitterness comprises a gum base, a sweetener and a cooling composition comprising Structure I, II, or III

In another embodiment of the invention an ingestible product for humans or animals which comprises a product base selected from the group consisting of baked goods, dairy products, fruit ices, confectionery products, sugarless candies, jams, jellies, gelatins, puddings, animal feeds, pressed confectionery tablets, hard-boiled candies, pectin-based candies, chewy candies, crème-centered candies, fondants, sugarless hard-boiled candies, sugarless pectin-based candies, sugarless chewy candies, sugarless crème-centered candies, toothpastes, mouthwashes, breath fresheners, carbonated beverages, mineral waters, powdered beverage mixes, other non-alcoholic beverages, cough drops, lozenges, cough mixtures, decongestants, anti-irritants, antacids, anti-indigestion preparations and oral analgesics, and an effective amount of Structure I, II or III.

Another embodiment of the invention is directed to a process for augmenting, enhancing or imparting a taste or cooling or refreshing effect in or to a cosmetic, skin care product, lip gloss, hair care product, cologne, shaving balm, after-shave lotion, dairy product, fruit ice preparation, confectionery, mouth and throat lozenges, cough mixtures, decongestants, antacids, oral analgesics or other medicinal products, chewing gum, candy (including hard candy and soft candy), fondants, toothpaste, mouthwashes, mineral water, alcoholic beverage, non-alcoholic beverage, powdered beverage, or other foodstuff comprising the step of adding to a cosmetic, skin care product, lip gloss, hair care product, cologne, shaving balm, after-shave lotion, dairy product, fruit ice preparation, confectionery, lozenges, cough mixtures, decongestants, antacids, oral analgesics or other medicinal product, chewing gum, candy, fondants, toothpaste, mouthwashes, mineral water, alcoholic beverage, non-alcoholic beverage, powdered beverage, or other foodstuff, a taste or cooling or refreshing effect augmenting, enhancing or imparting quantity and concentration of Structure I, Structure II or Structure III.

Structure I, Structure II and Structure III are known by one skilled in the art as 1-tert-butyl-N-[4-(cyanomethyl)phenyl]cyclohexanecarboxamide, 1-tert-butyl-N-[2-(pyridine-2-yl)ethyl]cyclohexanecarboxamide, 1-tert-butyl-N-[(pyridine-2-yl)methyl]cyclohexanecarboxamide, respectively.

Structure I is described having a bitter upfront, intense cooling upon expectorating flavor properties when tasted in water at 10 ppm.

Structure II is described having a honey, cream soda, slightly bitter, cooling flavor properties after about thirty seconds when tasted in water at 10 ppm.

Upon first tasting Structure III did not have much cooling except on breath-in. After expectorating, cooling began to build over time. The residual cooling (approx. five minutes) after expectorating earned a rating of 4 while when the cooler was in mouth, it was rated a 1 (on a scale of 1 to 9) when tasted in water at 10 ppm.

Based on this flavor evaluation, it is indicative that the Structure I, Structure II and Structure III of our invention are useful for augmenting, enhancing or imparting a taste or cooling or refreshing effect in or to a cosmetic, skin care product, lip gloss, hair care product, cologne, shaving balm, after-shave lotion, dairy product, fruit ice preparation, confectionery, mouth and throat lozenges, cough mixtures, decongestants, antacids, oral analgesics or other medicinal products, chewing gum, candy (including hard candy and soft candy), fondants, toothpaste, mouthwashes, mineral water, alcoholic beverage, non-alcoholic beverage, powdered beverage, or other foodstuff as a result of adding to a cosmetic, skin care product, lip gloss, hair care product, cologne, shaving balm, after-shave lotion, dairy product, fruit ice preparation, confectionery, lozenges, cough mixtures, decongestants, antacids, oral analgesics or other medicinal product, chewing gum, candy, fondants, toothpaste, mouthwashes, mineral water, alcoholic beverage, non-alcoholic beverage, powdered beverage, or other foodstuff, a taste or cooling or refreshing effect augmenting, enhancing or imparting quantity and concentration of Structure I, Structure II and Structure III.

As used herein the term, olfactory effective amount is understood to mean the amount of compound in flavor compositions, oral care compositions and articles, nasal care compositions and articles, skin care compositions, hair care compositions, cosmetic compositions, and other consumable materials as defined herein, the individual component will contribute to its particular olfactory characteristics, but the flavor, taste and aroma effect on the overall composition will be the sum of the effects of each of the cooling and/or refreshing and/or pungent flavor and/or sense imparting, augmenting or enhancing ingredients. As used herein taste effects include cooling, refreshing and pungent effects. Thus the compounds of the invention can be used to alter the taste characteristics of the flavor composition by modifying the taste reaction contributed by another ingredient in the composition. The amount will vary depending on many factors including other ingredients, their relative amounts and the effect that is desired.

The preferred usage level of Structure I, Structure II and Structure III used in products is at a level from about 0.001 to about 1 weight percent (%) and more preferably 0.015 to about 0.15 weight percent and most preferably about 0.04 weight percent.

The usage levels of Structure I, Structure II and Structure III vary depending on the product in which the compounds are employed.

Thus, with reference to the use of Structure I, Structure II and Structure III of our invention in alcoholic beverages, the usage level is from about 0.0005 to about 0.02 weight percent, preferably from about 0.002 to about 0.0150 weight percent and most preferably from about 0.0030 to about 0.0080 weight percent.

With reference to the use of the Structure I, Structure II and Structure III of our invention in non-alcoholic beverages including carbonated beverages and fruit drinks, the non-alcoholic beverages are flavored at levels of from about 0.0001 to about 0.0030 weight percent, preferably from about 0.0005 to about 0.0015 weight percent.

With reference to the use of the Structure I, Structure II and Structure III of our invention in toothpaste, the toothpaste can be satisfactorily flavored by using Structure I, Structure II and Structure III at levels of from about 0.02 to about 0.07 weight percent, more preferably from about 0.03 to about 0.06 weight percent and most preferable from about 0.035 up to about 0.055 weight percent.

With reference to the use of the Structure I, Structure II and Structure III of our invention in candy products including hard candy, the candy can be flavored at levels of from about 0.05 to about 0.25 weight percent; preferably from about 0.1 to about 0.2 weight percent.

With reference to the use of the Structure I, Structure II and Structure III of our invention in chewing gum, chewing gum usage levels are from about 0.01 to about 1 weight percent, preferably from about 0.1 to about 0.5 weight percent and more preferably from about 0.15 to about 0.25 weight percent.

When used in combination with other cooling compounds and compositions one skilled in the art may modify the levels to achieve the desired result.

When used in combination with other cooling compounds and compositions Structure I, Structure II and Structure III may be used at a level from about 0.01 to about 0.2 weight percent.

The term foodstuff as used herein includes both solid and liquid ingestible materials for man or animals, which materials usually do, but need not, have nutritional value. Thus, foodstuffs include meats, gravies, soups, convenience foods, malt, alcoholic and other beverages, milk and dairy products, seafood, including fish, crustaceans, mollusks and the like, candies, vegetables, cereals, soft drinks, snacks, dog and cat foods, other veterinary products and the like.

When Structure I, Structure II and Structure III is used in a flavoring composition, they can be combined with conventional flavoring materials or adjuvants. Such co-ingredients or flavor adjuvants are well known in the art for such use and have been extensively described in the literature. Requirements of such adjuvant materials are: (1) that they be non-reactive with Structure I, Structure II and Structure III; (2) that they be organoleptically compatible with the Structure I, Structure II and Structure III of our invention whereby the flavor of the ultimate consumable material to which Structure I, Structure II and Structure III is added is not detrimentally affected by the use of the adjuvant; and (3) that they be ingestible acceptable and thus nontoxic or otherwise non-deleterious. Apart from these requirements, conventional materials can be used and broadly include other flavor materials, vehicles, stabilizers, thickeners, surface active agents, conditioners and flavor intensifiers.

Such conventional flavoring materials include saturated fatty acids, unsaturated fatty acids and amino acids; alcohols including primary and secondary alcohols, esters, carbonyl compounds including ketones, other than the menthyl half acid esters or N-alkyl alkamic acid menthyl esters of our invention and aldehydes; lactones; other cyclic organic materials including benzene derivatives, acyclic compounds, heterocyclics such as furans, pyridines, pyrazines and the like; sulfur-containing compounds including thiols, sulfides, disulfides and the like; proteins; lipids, carbohydrates; so-called flavor potentiators such as monosodium glutamate; magnesium glutamate, calcium glutamate, guanylates and inosinates; natural flavoring materials such as hydrolyzates, cocoa, vanilla and caramel; essential oils and extracts such as anise oil, clove oil and the like and artificial flavoring materials such as vanillin, ethyl vanillin and the like.

Specific preferred flavor adjuvants include but are not limited to the following: anise oil; ethyl-2-methyl butyrate; vanillin; cis-3-heptenol; cis-3-hexenol; trans-2-heptenal; butyl valerate; 2,3-diethyl pyrazine; methyl cyclo-pentenolone; benzaldehyde; valerian oil; 3,4-dimeth-oxyphenol; amyl acetate; amyl cinnamate; γ-butyryl lactone; furfural; trimethylpyrazine; phenyl acetic acid; isovaleraldehyde; ethyl maltol; ethyl vanillin; ethyl valerate; ethyl butyrate; cocoa extract; coffee extract; peppermint oil; spearmint oil; clove oil; anethol; cardamom oil; wintergreen oil; cinnamic aldehyde; ethyl-2-methyl valerate; γ-hexenyl lactone; 2,4-decadienal; 2,4-heptadienal; methyl thiazole alcohol (4-methyl-5-β-hydroxyethyl thiazole); 2-methyl butanethiol; 4-mercapto-2-butanone; 3-mercapto-2-pentanone; 1-mercapto-2-propane; benzaldehyde; furfural; furfuryl alcohol; 2-mercapto propionic acid; alkyl pyrazine; methylpyrazine; 2-ethyl-3-methylpyrazine; tetramethylpyrazine; polysulfides; dipropyl disulfide; methyl benzyl disulfide; alkyl thiophene; 2,3-dimethyl thiophene; 5-methyl furfural; acetyl furan; 2,4-decadienal; guaiacol; phenyl acetaldehyde; β-decalactone; d-limonene; acetoin; amyl acetate; maltol; ethyl butyrate; levulinic acid; piperonal; ethyl acetate; n-octanal; n-pentanal; n-hexanal; diacetyl; monosodium glutamate; monopotassium glutamate; sulfur-containing amino acids, e.g., cysteine; hydrolyzed vegetable protein; 2-methylfuran-3-thiol; 2-methyldihydrofuran-3-thiol; 2,5-dimethylfuran-3-thiol; hydrolyzed fish protein; tetramethylpyrazine; propyl propenyl disulfide; propyl propenyl trisulfide; diallyl disulfide; diallyl trisulfide; dipropenyl disulfide; dipropenyl trisulfide; 4-methyl-2-[(methylthio)-ethyl]-1,3-dithiolane; 4,5-dimethyl-2-(methylthiomethyl)-1,3-dithiolone; and 4-methyl-2-(methylthiomethyl)-1,3-dithiolane. These and other flavor ingredients are provided in U.S. Pat. Nos. 6,110,520 and 6,333,180.

Structure I, Structure II and Structure III or compositions incorporating them, as mentioned above can be combined with one or more vehicles or carriers for adding them to the particular product. Vehicles can be water-soluble or oil-soluble; and can be edible or otherwise suitable materials such as ethyl alcohol, propylene glycol, water, triacetin, vegetable oil, triethyl citrate and the like, as described supra. Carriers include materials such as gum arabic, carrageenan, xanthan gum, guar gum and the like.

Structure I, Structure II and Structure III prepared according to our invention can be incorporated with the carriers by conventional means such as spray-drying, extrusion, drum-drying and the like. Such carriers can also include materials for coacervating the menthyl half-acid ester derivatives including menthyl half acid esters or N-alkyl alkamic acid menthyl esters of our invention to provide encapsulated products, as set forth supra. When the carrier is an emulsion, the flavoring composition can also contain emulsifiers such as mono- and diglycerides or fatty acids and the like. With these carriers or vehicles, the desired physical form of the compositions can be prepared.

The quantity of Structure I, Structure II and Structure III utilized should be sufficient to impart the desired flavor characteristic to the product, but on the other hand, the use of an excessive amount of Structure I, Structure II and Structure III is not only wasteful and uneconomical, but in some instances, too large a quantity may unbalance the flavor or other organoleptic properties of the product consumed. The quantity used will vary depending upon the ultimate foodstuff; the amount and type of flavor initially present in the foodstuff; the further process or treatment steps to which the foodstuff will be subjected; regional and other preference factors; the type of storage, if any, to which the product will be subjected; and the preconsumption treatment such as baking, frying and so on, given to the product by the ultimate consumer. Accordingly, the terminology effective amount and sufficient amount is understood in the context of the present invention to be quantitatively adequate to alter the flavor of the foodstuff.

It will be understood that the embodiments described herein are merely exemplary, and that one skilled in the art may make variations and modifications without departing from the spirit and scope of the invention. All such variations and modifications are intended to be included within the scope of the invention as described hereinabove. Further, all embodiments disclosed are not necessarily in the alternative, as various embodiments of the invention may be combined to provide the desired result. As used herein all percentages are weight percent unless otherwise noted, ppm is understood to stand for parts per million, mL is understood to be milliliter, g is understood to be gram, and mol is understood to be mole. Me is understood to stand for methyl, Et is understood to stand ethyl, Ac is understood to stand for an acetyl group. IFF as used in the examples is understood to mean International Flavors & Fragrances Inc., New York, N.Y., USA.

### EXAMPLE 1

### Structure I

### Synthesis of 1-tert-butyl-N-[4-(cyanomethyl)phenyl]cyclohexanecarboxamide

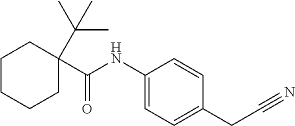

The 1-tert-Butylcyclohexane carboxylic acid used in this example was obtained according to literature publication “Cyclohexane derivatives. VIII. Preparation and mass spectra of the seven tert-butylcyclohexanecarboxylic acid” Recueil des Travaux Chimiques des Pays-Bas (1970), 89, (5), 521-534; C₁₁H₂₀O₂ (MW 184.28); ¹H NMR (500 MHz, CDCl₃) δ: 0.96 (s, 9H), 1.03-1.13 (m, 1H), 1.19-1.38 (m, 4H), 1.61-1.72 (m, 3H), 2.12 (d, 2H, J=12.11 Hz).

The 1-tert-butylcyclohexane carboxylic acid (3.5 g, 19.1 mmol) was heated under reflux with thionyl chloride (7 mL) for one hour. Then the excess of thionyl chloride was distilled off in vacuum. Residue was dissolved in CH₂Cl₂ (30 mL). This solution was added drop wise to the mixture of 4-Aminobenzyl cyanide (12.6 g, 95.4 mmol) and triethylamine (19.0 g, 180.0 mmol) at 0° C. After ten minutes the solution was allowed to warm to room temperature and then reaction was carry out in reflux for 12 hours. CH₂Cl₂ (50 mL) was added to the reaction and this mixture was washed by 0.1 N HCl aq, water, and by brine and dried over MgSO₄. The mixture was then filtrated and evaporated give crude solid which was purified by chromatographic column (hexane 90/ethyl acetate 10) providing 3 g (53%) of product as white solid.

C₁₉H₂₆N₂O (MW 298.4); ¹H NMR (500 MHz, CDCl₃) δ: 0.98 (s, 9H), 1.08-1.11 (m, 1H), 1.30-1.45 (m, 4H), 1.63-1.74 (m, 3H), 2.03-2.19 (m, 2H), 3.70 (s, 2H), 7.24 (d, 2H, J=8.33 Hz), 7.45 (br. s, 1H), 7.55 (d, 2H, J=8.40 Hz).

### EXAMPLE 2

### Structure II

### Synthesis of 1-tert-butyl-N-[2-(pyridine-2-yl)ethyl]cyclohexanecarboxamide

In the similar manner to the synthesis of 1-tert-butyl-N-[4-(cyanomethyl)phenyl]cyclohexanecarboxamide, 1-tert-butyl-N-[2-(pyridine-2-yl)ethyl]cyclohexane carboxamide was obtained starting from 1-tert-butylcyclohexane carboxylic acid.

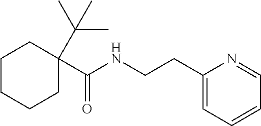

C₁₈H₂₈N₂O (MW 288.22); ¹H NMR (500 MHz, CDCl₃) δ: 0.86 (s, 9H), 1.02-1.08 (m, 1H), 1.16-1.24 (m, 2H), 1.26-1.30 (m, 2H), 1.50-1.59 (m, 3H), 2.02 (d, 2H, J=12.92 Hz), 3.03 (t, 2H, J=6.27 Hz), 6.79 (br. s, 1H), 7.14 (t, 1H, J=6.14 Hz), 7.19 (d, 1H, J=7.76 Hz), 7.61 (d, 1H, J=1.60 Hz, of t, J=7.64 Hz), 8.51 (d, 1H, J=4.68 Hz).

### EXAMPLE 3

### Structure III

### Synthesis of 1-tert-butyl-N-[(pyridine-2-yl)methyl]cyclohexanecarboxamide

In the similar manner to the synthesis of 1-tert-butyl-N-[4-(cyanomethyl)phenyl]cyclohexanecarboxamide, 1-tert-butyl-N-[(pyridine-2-yl)methyl]cyclohexanecarboxamide was obtained starting from 1-tert-butylcyclohexane carboxylic acid. During workup water was used instead 0.1N HCl aq.

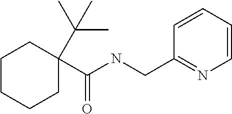

C₁₇H₂₆N₂O (MW 274.20); ¹H NMR (500 MHz, CDCl₃) δ: 0.92 (s, 9H), 1.02-1.13 (m, 1H), 1.25-1.38 (m, 4H), 1.57-1.66 (m, 3H), 2.09-2.14 (m, 2H), 4.60 (d, 2H, J=4.75 Hz), 6.98 (br. s, 1H), 7.18 (t, 1H, J=5.14 Hz), 7.30 (d, 1H, J=7.83 Hz), 7.65 (t, 1H, J=7.62 Hz, of d, J=1.53 Hz), 8.53 (d, 1H, J=4.59 Hz).

### EXAMPLE 4

In this example, Structure III was mixed with a fondant, commercially available from Confectionary House, Troy, N.Y., at levels listed in Table 1.

### Evaluations

TABLE I

| COOLER | FONDANT | COMMENTS |
|---|---|---|
| Structure III | 50 ppm | Slight cooling but quick onset, fills mouth |
| | 100 ppm | Quick onset slight bitterness perceived |
| | 200 ppm | Similar to 100 ppm but very bitter |

### EXAMPLE 5

The following gum base formulation was prepared:

TABLE II

| Ingredients | Parts by Percentage (%) | Parts by Weight (grams) |
|---|---|---|
| Gum Base, Hades-T | 29.35 | 205.45 |
| Maltitol Syrup | 3.00 | 21 |
| Sorbitol Powder | 48.30 | 338.1 |
| Mannitol Powder | 8.00 | 56 |
| Glycerin | 9.00 | 63 |
| Sucralose | 0.20 | 1.4 |
| Acesulfame K | 0.15 | 1.05 |
| Flavor | 1.00 | 7 |
| Total | 99.00 | 693 |

The orange flavor is commercially available from IFF. The gum base, Hades-T is commercially available from Cafosa Gum, Barcelona Spain.

Structure III by itself at levels detailed in the Table III was blended with the gum base in a Sigma mixer. The resultant chewing gum blend was then manufactured into strips 1 inch in width and 0.1 inches in thickness. These strips were cut into lengths of 3 inches each. A control gum, without any coolers, was also manufactures into strips and this control gum exhibited a flavor without imparting any cooling effect.

The resulting gum blends had a substantially identical to the taste profile of the control gum; however the gum blends had cooling effects as described below.

Samples were profiled by a highly trained descriptive analysis panel. The ballot consisted of the following terms: Total impact, total mint, cooling-mouth closed, cooling—breath in, nasal cooling—breathe in, throat irritation, bitterness, tongue irritation, numbing. Each attribute was rated at discrete time points including 1 minute, 3 minutes, 5 minutes, 10 minutes, 20 minutes, and 30 minutes.

TABLE III

| COMPOUND | LEVEL | Time and Evaluation |
|---|---|---|
| Structure III | 0.20% | 0-1 minute |
| | | Sweet, citrus. Not cooling slightly bitter |
| | | 1-3 minutes |
| | | Cooling is perceivable, but weak. |
| | | 3-5 minutes |
| | | More cooling. Cooling when breathing. Little |
| | | bitterness., cooling very weak |
| | | 5-10 minutes |
| | | Cooling is all over the mouth. Some bitterness |
| | | is perceivable, very slight cooling again very |
| | | weak. |
| | | 10-20 minutes |
| | | Cooling is all over the mouth but not very |
| | | intensive., little to no cooling |
| | | 20-30 minutes |
| | | More bitterness is perceivable. Cooling is |
| | | moderate and slowly starting to decline. |
| | | little to no cooling |

What is claimed:

1. A compound: 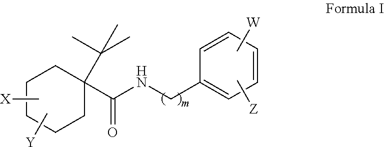
wherein X, Y is independently selected from a C1-C4 straight or branched alkyl or H atom; and m is 0, 1 or 2, Z when present is nitrogen, W and W′ are selected independently from the group consisting of H, OH, C1-C4 straight or branched alkyl, and a C1-C4 straight or branched alkoxy or W is group (CH₂)n-R, where n is 0 or 1 and R is selected from OH, OMe, OEt, NO₂, CN, Ac, SO₂NH₂, CHO, CO₂H, CONH₂, CONHR², where R² is selected from C1-C4, and C1-C4 alkyl carboxylates such as CO₂Me or CO₂Et.

2. The compound of claim 1 wherein the compound is selected from the group consisting of

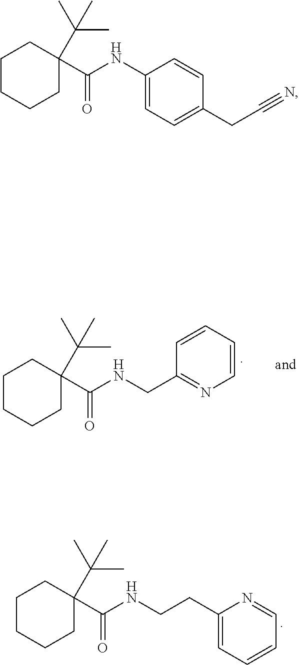

3. A composition selected from topical products for humans and animals, oral care products, nasal care products, and chewing gum which comprises a product base and an effective amount of the compound of claim 1.

4. A composition as claimed in claim 1 which comprises from 0.001-1.0% by weight, based on the total weight of the composition, of said coolant.

5. A composition comprising a product base selected from the group consisting of baked goods, dairy products, fruit ices, confectionery products, sugarless candies, jams, jellies, gelatins, puddings, animal feeds, pressed confectionery tablets, hard-boiled candies, pectin-based candies, chewy candies, crème-centered candies, fondants, sugarless hard-boiled candies, sugarless pectin-based candies, sugarless chewy candies, sugarless crème-centered candies, toothpastes, mouthwashes, breath fresheners, carbonated beverages, mineral waters, powdered beverage mixes, other non-alcoholic beverages, cough drops, lozenges, cough mixtures, decongestants, anti-irritants, antacids, anti-indigestion preparations and oral analgesics, and an effective amount of a compound: 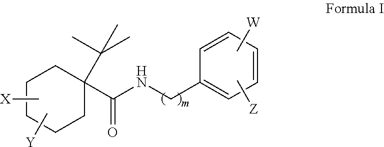
wherein X, Y is independently selected from a C1-C4 straight or branched alkyl or H atom; and m is 0, 1 or 2, Z when present is nitrogen, W and W′ are selected independently from the group consisting of H, OH, C1-C4 straight or branched alkyl, and a C1-C4 straight or branched alkoxy or W is group (CH₂)n-R, where n is 0 or 1 and R is selected from OH, OMe, OEt, NO₂, CN, Ac, SO₂NH₂, CHO, CO₂H, CONH₂, CONHR², where R² is selected from C1-C4, and C1-C4 alkyl carboxylates such as CO₂Me or CO₂Et.

6. The composition of claim 5 wherein the compound is selected from the group consisting of

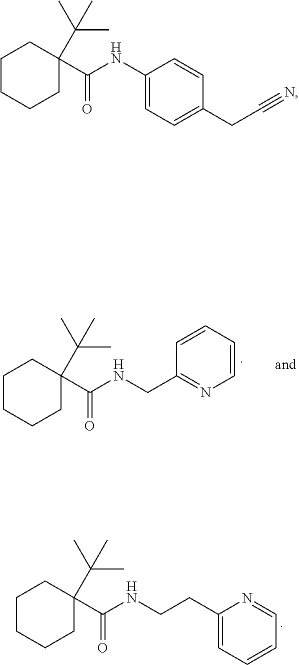

7. A composition as claimed in claim 5 wherein the orally ingested products are selected from lozenges, cough mixtures, decongestants, anti-irritants, antacids, anti-indigestion preparations and oral analgesics.

8. A composition as claimed in claim 5 wherein the ingestible product is a foodstuff selected from the group consisting of baked goods, dairy products, fruit ices, confectionery products, sugarless candies, jams, jellies, gelatins, puddings and animal feeds.

9. A composition as claimed in claim 5 wherein the topical product is selected from the group consisting of face creams, talcum powders, hair oils, shampoos, bath oils, bath salts, toilet soaps, colognes, antiperspirants, toilet water, perfume, shaving lotion, shaving cream, hair tonic, ointments and lotions.

10. A composition as claimed in claim 5 wherein the oral care product is selected from dentifrices and mouthwashes.

11. A composition as claimed in claim 5 wherein the ingestible product is cough drops.

12. A composition as claimed in claim 5 which comprises from 0.001-1.0% by weight, based on the total weight of the composition, of said coolant.

13. A composition as claimed in claim 5 wherein said composition is selected from pressed confectionery tablets, hard boiled candies, pectin-based candies, chewy candies, cream-centered candies, fondants, sugarless hard-boiled candies, sugarless pectin-based candies, sugarless chewy candies, sugarless cream-centered candies, animal feeds, breath fresheners, carbonated beverages, mineral waters, powdered beverage mixes and non-alcoholic beverages.

14. A process for augmenting, enhancing or imparting a taste or cooling or refreshing effect in or to a cosmetic, skin care product, lip gloss, hair care product, cologne, shaving balm, after-shave lotion, dairy product, fruit ice preparation, confectionery, mouth and throat lozenges, cough mixtures, decongestants, antacids, oral analgesics or other medicinal products, chewing gum, candy, fondants, toothpaste, mouthwashes, mineral water, alcoholic beverage, non-alcoholic beverage, powdered beverage, or other foodstuff comprising the step of adding to a cosmetic, skin care product, lip gloss, hair care product, cologne, shaving balm, after-shave lotion, dairy product, fruit ice preparation, confectionery, lozenges, cough mixtures, decongestants, antacids, oral analgesics or other medicinal product, chewing gum, candy, fondants, toothpaste, mouthwashes, mineral water, alcoholic beverage, non-alcoholic beverage, powdered beverage, or other foodstuff, a taste or cooling or refreshing effect augmenting, enhancing or imparting quantity and concentration of Formula I: 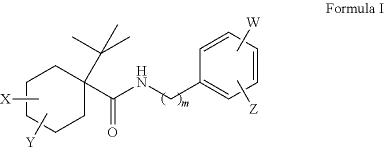
wherein X, Y is independently selected from a C1-C4 straight or branched alkyl or H atom; and m is 0, 1 or 2, Z when present is nitrogen, W and W′ are selected independently from the group consisting of H, OH, C1-C4 straight or branched alkyl, and a C1-C4 straight or branched alkoxy or W is group (CH₂)n-R, where n is 0 or 1 and R is selected from OH, OMe, OEt, NO₂, CN, Ac, SO₂NH₂, CHO, CO₂H, CONH₂, CONHR², where R² is selected from C1-C4, and C1-C4 alkyl carboxylates such as CO₂Me or CO₂Et.

15. The process of claim 14 wherein the compound is selected from the group consisting of

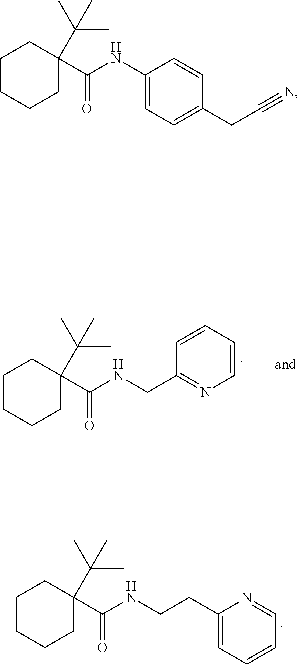

16. A chewing gum composition comprising a gum base, a sweetener and an effective amount of Formula I: 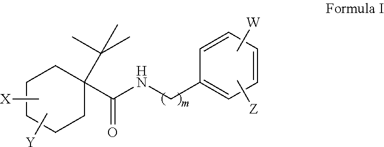
wherein X, Y is independently selected from a C1-C4 straight or branched alkyl or H atom; and m is 0, 1 or 2, Z when present is nitrogen, W and W′ are selected independently from the group consisting of H, OH, C1-C4 straight or branched alkyl, and a C1-C4 straight or branched alkoxy or W is group (CH₂)n-R, where n is 0 or 1 and R is selected from OH, OMe, OEt, NO₂, CN, Ac, SO₂NH₂, CHO, CO₂H, CONH₂, CONHR², where R² is selected from C1-C4, and C1-C4 alkyl carboxylates such as CO₂Me or CO₂Et.

17. The chewing gum composition of claim 16 wherein the compound is selected from the group consisting of [chemical structure]
